(19) United States
(12) Patent Application Publication
(10) Pub. No.: US 2010/0022774 A1
(43) Pub. Date: Jan. 28, 2010

(54) PROCESS USEFUL IN THE PREPARATION OF MORPHINAN ANTAGONISTS

(76) Inventors: Ole Heine Kvernenes, Oslo (NO); Anne Mette Nygard, Jar (NO); Audun Heggelund, Oslo (NO); Harald Halvorsen, Oslo (NO)

Correspondence Address: G Kenneth Smith, 1645 Briarwood Circle, Bethlehem, PA, 18015, US

(21) Appl. No.: 12/300,055

(22) PCT Filed: May 25, 2007
(86) PCT No.: PCT/EP2007/004679

§ 371 Date: Sep. 25, 2009

(30) Foreign Application Priority Data
May 25, 2006 (GB) 0610385.7
May 25, 2006 (GB) 0610386.5
May 25, 2006 (GB) 0610387.3
May 25, 2006 (GB) 0610388.1

(51) Int. Cl. C07D 489/00

(52) U.S. Cl. 546/45

(57) ABSTRACT

A process for the preparation of morphinan antagonists of Formula (I) is described wherein R is a cyclopropyl, cyclobutyl or vinyl group and X is O, CH₂ or diC₁₋₄alkoxy group (optionally linked).

The present invention relates to a process for the preparation of naltrexone or naloxone or derivatives or salts thereof.

It has been known for over 30 years that when suitable substituents are introduced on the nitrogen atom of a morphinan derivative, the resulting compounds are narcotic antagonists that may also have analgesic properties and are not addictive.

Some commercial and well known morphinan antagonists are shown below. These include naltrexone and naloxone.

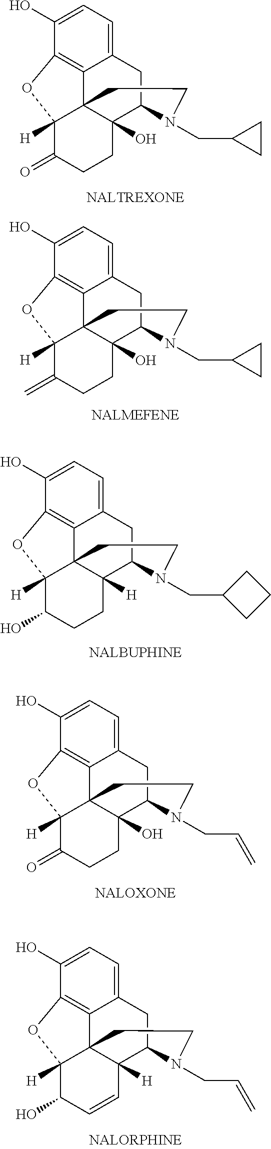

Numerous reaction sequences are known for the preparation of naltrexone and naloxone but these generally involve numerous steps and can lead to low overall yields. It is still desirable to find a method of producing naltrexone and naloxone which can start from readily commercially available compounds and lead to good yields of naltrexone and naloxone by processes that do not involve too many individual reactions. A reaction sequence that commences from oxycodone and leads to the desired compounds in an effective manner is now provided. The route employed offers the potential advantage of requiring fewer steps.

The present invention provides a process for the preparation of a compound of the formula (I), or a pharmaceutically acceptable salt thereof, for example the HCl salt:

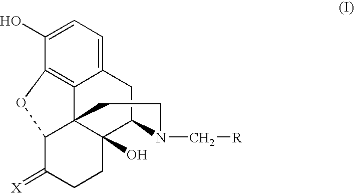

wherein R is a cyclopropyl, cyclobutyl or vinyl group and X is O, CH₂ or diC₁₋₄alkoxy group (optionally linked), which process comprises:
(i) the N-demethylation and optionally the O-demethylation of a compound of the formula (II):

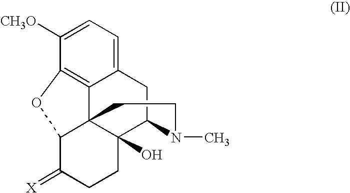

wherein X is as defined in relation for formula (I) to yield a compound of the formula (III) or a pharmaceutically acceptable salt thereof, for example the HCl salt:

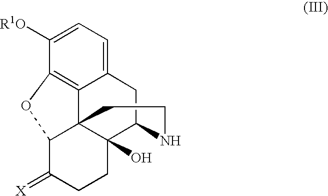

wherein X is as defined in relation to formula (I) and R¹ is CH₃ if only the N-demethylation step is carried out or is H if both the N- and O-demethylation steps are carried out; followed by
(ii) reaction of the compound of formula (III) or a pharmaceutically acceptable salt thereof, with either
   (a) a compound of the formula R—CHO where R is as defined in relation to formula (I), followed by reduction of the iminium ion double bond of the resulting intermediate, or
   (b) a compound of the formula Q-CH₂R wherein R is as defined in relation to the compound of formula (I) and Q is Cl, Br,OSO₂PhMe or OSO₂Me;
to form a compound of the formula (IV) or a pharmaceutically acceptable salt thereof, for example a HCl salt:

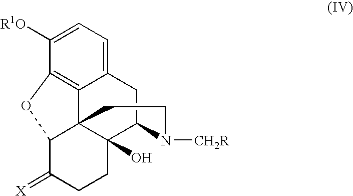

wherein X and R are as defined in relation to the compound of the formula (I) and R¹ is defined as in relation to the compound of formula (III);
(iii) and if R¹ is CH₃, followed by reaction of the compound of formula (IV) or a pharmaceutically acceptable salt thereof with BBr₃ or other reagents capable of demethylating an aryl methyl ether; and optionally
(iv) obtaining the free base or pharmaceutically acceptable salt thereof as desired.

The free base of the compound of formula (I) may be obtained by neutralisation of a salt of the compound of formula (I). A pharmaceutically acceptable salt of the compound of formula (I) may be obtained by mixing the free base or a salt of the compound of formula (I) with the appropriate acid.

A preferable embodiment of the invention is the following process for the preparation of a compound of the formula (I), optionally in the form of a pharmaceutically acceptable salt, for example the HCl salt:

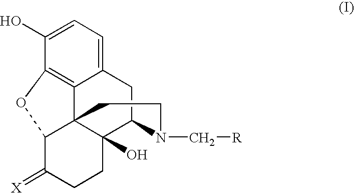

wherein R is a cyclopropyl, cyclobutyl or vinyl group and X is O, CH₂ or diC₁₋₄alkoxy group (optionally linked), which process comprises:
(i) the N-demethylation of a compound of the formula (II):

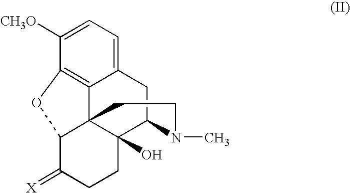

wherein X is as defined in relation for formula (I) by reaction with a-chloroethylchloroformate to yield a compound of the formula (III) or a pharmaceutically acceptable salt thereof, for example the HCl salt:

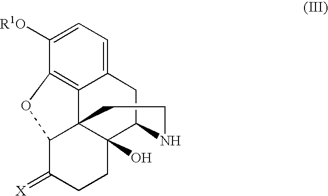

wherein X is as defined in relation to formula (I) and R¹ is CH₃, followed by
(ii) reaction of the compound of formula (III) or a pharmaceutically acceptable salt thereof, with either
   (a) a compound of the formula R—CHO where R is as defined in relation to formula (I), followed by reduction of the iminium ion double bond of the resulting intermediate, or
(b) with a compound of the formula Q-CH₂R wherein R is as defined in relation to the compound of formula (I) and Q is Cl, Br, OSO₂PhMe or OSO₂Me;
to form a compound of the formula (IV) or a pharmaceutically acceptable salt thereof, for example a HCl salt:

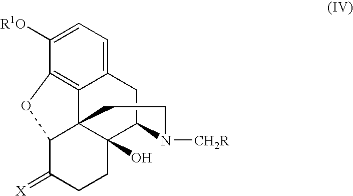

wherein X and R are as defined in relation to the compound of the formula (I) and R¹ is CH₃;
followed by:
(iii) reaction of the compound of formula (IV) or a pharmaceutically acceptable salt thereof, with BBr₃ or other reagents capable of demethylating an aryl methyl ether, and optionally
(iv) obtaining the free base or pharmaceutically acceptable salt thereof as desired.

Favourably X is O, CH₂ or a diC₁₋₆ alkoxy group such as a OCH₂CH₂O group. Most aptly X is O or OCH₂CH₂O. More preferably X is O.

Favourably R is cyclopropyl or a vinyl group. Preferably R is cyclopropyl.

If the final product which is required is one which contains the 6-keto group, the preceding reaction sequence can be performed on compounds wherein X is O. Alternatively, the preceding reaction sequence can be performed on compounds wherein X is diC₁₋₆alkoxy (optionally liked), for example a OCH₂CH₂O group, which can be converted to the 6-keto group by standard methods, for example hydrolysis under mildly acidic conditions.

N-demethylation Reaction

The N-demethylation reaction results in an easily hydrolysable carbamate intermediate. Hydrolysis of the carbamate intermediate, for example with MeOH, aqueous THF or aqueous isopropanol results in the N-demethylated product.

The N-demethylation reaction is most suitably performed in an aprotic solvent such as dichloromethane, 1,2-dichloroethane, dimethylformamide, acetonitrile tetrahydrofuran or the like. A favoured solvent is dichloromethane. Surprisingly, a most preferable solvent is acetonitrile.

The N-demethylation is preferably carried out in the presence of a proton acceptor such as carbonates or bicarbonates. A particularly suitable proton acceptor is anhydrous sodium carbonate. It has surprisingly been found that if the proton acceptor is added to the reaction mixture in more than one portion, for example in two or three separate portions, higher yields can be obtained.

Suitably, the temperature of the N-demethylation reaction is generally non-extreme, for example commencing at and carrying out at an ambient temperature (about 20-25° C.). Optionally the temperature may be progressed to about 40° C., for example under reflux in dichloromethane.

It is preferred to exclude water, for example by carrying out the reaction under nitrogen.

The reaction time may be significantly reduced with the addition of phase transfer catalysts, such as for example, tetrabutylammonium bromide (TBAB), hexadecyltrimethyl ammonium bromide, methyltrioctyl ammonium chloride, benzyltributyl ammonium chloride and tetrabutyl ammonium bisulfate. A preferable phase transfer catalyst is tetrabutylammonium bromide (TBAB)

N-alkylation Reaction

By Reductive Alkylation (Route (a))

The conversion of the compound of the formula (III) (or a pharmaceutically acceptable salt thereof) into a compound of the formula (IV) (or a pharmaceutically acceptable salt thereof) by reaction with R—CHO (VI) may be best effected at a depressed temperature especially when X is O. Somewhat higher temperatures may be employed when X is a protected CO group. When X is O the reaction temperature of the reduction may be below −20°, for example at −30° C., although in the initial phase when the carboxaldehyde reacts with the secondary amine the temperature may be higher, for example at ambient temperature.

Suitable reducing reagents when X is O include triacetoxyborohydrides such as sodium triacetoxyborohydride, or a cyanoborohydride such as sodium cyanoborohydride. Hydrogen and a catalyst such as palladium may also be employed.

If X is a protected keto group or a CH₂ group more vigorous reducing agents may be employed, for example borohydrides such as sodium borohydride or other hydride reducing agents such as lithium aluminium hydride. Other suitable reducing agents include triethylsilane and phenylsilane.

The reaction may be carried out in a solvent such as tetrahydrofuran, ethanol, isopropanol, dimethylformamide, dimethylsulfoxide, dichloromethane, 1,2-dichloroethane or the like. A preferred solvent is 1,2-dichloroethane.

The reaction may be performed in high dilution, for example at least 20 ml of solvent per 100 mg of starting material.

By Direct N-alkylation (Route (b))

The conversion of the compound of the formula (III) (or a pharmaceutically acceptable salt thereof) into compound of formula (IV) (or a pharmaceutically acceptable salt thereof) by direct alkylation using a compound of the formula Q-CH₂R may be best effected at elevated temperatures, such as 40 to 100° C., 50 to 90° C., 50 to 85° C., 60 to 80° C. and preferably 70 to 80° C.

Suitable alkylating reagents include compounds of the following formula Q-CH₂—R wherein R is as defined in relation to the compound of formula (I) and Q is Cl, Br, OSO₂PhMe or OSO₂Me. Preferable reagents are alkyl halides, such akylhalides include cyclobutylmethyl bromide, cyclobutylmethyl chloride, allylbromide, and allylchloride. Most aptly, the reagent is cyclopropylmethylbromide.

The reaction may be carried out in a solvent such as tetrahydrofuran, ethanol, isopropanol, dimethylformamide, 1-2-dichloroethane and acetonitrile. A preferred solvent is acetonitrile.

The reaction may be carried out in the presence of a phase transfer catalyst, for example a crown ether. Examples of crown ethers include 15-crown-5 and 18-crown-6. A preferred crown ether is 18-Crown-6.

This reaction may be carried out at a low dilution, for example, 0.1-0.3M. A preferred concentration is between 0.2M and 0.3M, for example, 0.25M.

The reaction may also, for example, be carried out in the presence of potassium hydrogen carbonate, sodium hydrogen carbonate, potassium carbonate and sodium carbonate, preferably potassium hydrogen carbonate.

After work up and precipitation, compound (IV) (or a pharmaceutically acceptable salt thereof) is typically isolated in 85% yield and 96.5% purity.

O-demethylation Reaction

The reaction of the compound of formula (IV) (or a pharmaceutically acceptable salt thereof) with BBr₃ may take place in an aprotic solvent such as toluene, tetrahydrofuran, chloroform, dichloromethane, 1,2-dichloroethane or the like, preferably dichloromethane. The BBr₃ is generally added at a depressed temperature for example 0° C. to −20° C.

Generally, an ambient temperature, for example 20-30° C., is employed thereafter. After the reaction is complete, which generally takes from 2 to 18 hours (or 2 to 4 hours), it is quenched by using water containing a base such as ammonium hydroxide or sodium hydrogencarbonate, preferably at a depressed temperature, for example by using ice. The desired compound of formula (I) may be obtained from the organic phase.

The preceding compounds may be converted into salts if required by addition of the appropriate acid, for example ethanoic, lactic, benzoic, methanesulphonic, toluenesulphonic, mandelic, malic, hydrochloric, sulphuric, phosphoric acid or the like.

Normally, BBr₃ is slowly added to a solution of the compound of formula (IV). Alternatively, the solution a solution of the compound of formula (IV) can be added to a solution of BBr₃. For example a solution of the compound of formula (IV) may be added to a 0.5M solution of BBr₃ in DCM at, for example −10 to −30° C.

Alternatively, a further preferred embodiment of the invention is a process for the preparation of naltrexone or naloxone or a salt thereof which process comprises:

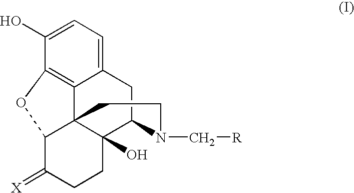

wherein R is a cyclopropyl, cyclobutyl or vinyl group and X is O, CH₂ or diC₁₋₄alkoxy group (optionally linked), which process comprises:

(i) the N-demethylation and O-demethylation of a compound of the formula (II):

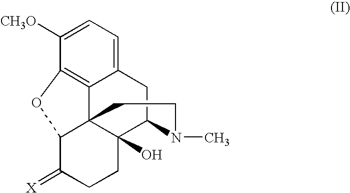

wherein X is as defined in relation for formula (I) to yield a compound of the formula (III) or a pharmaceutically acceptable salt thereof, for example the HCl salt:

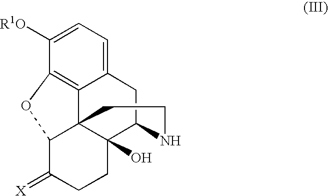

wherein X is as defined in relation to formula (I) and R¹ is H, followed by
(ii) reaction of the compound of formula (III) or a pharmaceutically acceptable salt thereof, with either
   (a) a compound of the formula R—CHO where R is as defined in relation to formula (I), to yield an intermediate containing the ion of formula (VII):

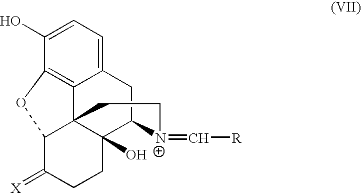

followed by reduction of the iminium ion double bond of the resulting intermediate (VII); or
(b) with a compound of the formula Q-CH₂R wherein R is as defined in relation to the compound of formula (I) and Q is Cl, Br, OSO₂PhMe or OSO₂Me, and optionally
(iii) obtaining the free base or pharmaceutically acceptable salt thereof as desired.

Most aptly X is O or OCH₂CH₂O. Preferably X is O.

O-demethylation and N-demethylation Reaction Steps

The conversion of compound of formula (II) into the compound of formula (III) (or a pharmaceutically acceptable salt thereof) may be effected in one or two steps.

Thus, for example, the compound of formula (II) may be reacted with a reagent which results in O-demethylation followed by reaction with a reagent which results in N-demethylation. Alternatively, the compound of formula (II) may be reacted with a reagent which results in N-demethylation followed by reaction with a reagent which results in O-demethylation. In one aspect the reagent employed effects both O-demethylation and N-demethylation.

In some methods of N-demethylation an intermediate carbamate is formed (for example from reaction with a chloroformate) which is then cleaved, for example by the use of lithium selectride (L-selectride).

If the chloroformate used is a-chloroethylchloroformate (ACE-Cl), the carbamate formed will be easily hydrolysed. Weak hydrolysing agents, such as MeOH may be used to hydrolyse these carbamates.

If the chloroformate used is C₂₋₆chloroformate, for example ethylchloroformate, the carbamate intermediate will be more stable. A suitable hydrolysing agent that can be used is lithium selectride (lithium tri-sec-butylborohydride). Another example of a chloroformate is phenyl chloroformate.

A suitable reagent of use in the O-demethylation and N-deprotection of carbamate intermediates of the compound of the formula (II) is lithium selectride (L-selectride).

An apt reagent for use for the O-demethylation of a compound of formula (II) is BBr₃. An apt reagent for use for the N-demethylation of a compound of formula (II) is a chloroformate, for example a-chloroC₁₋₆alkylchloroformate, preferably a-chloroethylchloroformate. A suitable reagent for use in the O-demethylation and N-demethylation (via a decarboxymethylation) of a compound of formula (II) is lithium selectride (tri-sec-butylborohydride).

The reaction of the compound of formula (II) (or its N-demethylated analogue) with BBr₃ may take place in an aprotic solvent such as tetrahydrofuran, chloroform, dichloromethane, 1,2-dichloroethane or the like. Addition of BBr₃ generally takes place at a depressed temperature for example 0° C. to −20° C. Generally, ambient temperature, for example 20-30° C., is thereafter employed.

After the reaction is complete, it is quenched by using water containing a base such as ammonium hydroxide or sodium bicarbonate, preferably at a depressed temperature, for example by using ice. The desired N and O-demethylated compound of formula (III) or a pharmaceutically acceptable salt thereof (i.e. R¹═H) may be obtained from the aqueous phase (whereas the N-methylated analogue of the compound of formula (III) or a pharmaceutical acceptable salt thereof (i.e. R¹═CH₃) may be obtained form the organic phase).

The reaction of a compound of formula (II) (or its O-demethyl analogue) with a chloroformate such as a-chloroethylchloroformate may be employed to effect N-demethylation.

The N-demethylation of a compound of formula (II) (or its O-demethylated analogue) into a compound of the formula (III) (or a pharmaceutically acceptable salt thereof) may be effected at non-extreme elevated temperature, for example at the reflux point of the solvent employed or at about 20-70° C., 30-70° C., for example 40-50° C. or favourably 20-25° C.

The solvent employed may be carried out in a solvent such as tetrahydrofuran, acetonitrile, dimethylformamide, dichloromethane, 1,2-dichloroethane or the like. A favoured solvent is dichloromethane. Surprisingly a most preferably solvent is acetonitrile.

Generally, the reaction is performed under anhydrous conditions, for example under nitrogen. The reaction generally employs a proton abstracting agent, for example carbonate or bicarbonate. Anhydrous sodium carbonate is particularly apt.

The reaction time may be significantly reduced with the addition of phase transfer catalysts, such as for example, tetrabutylammonium bromide hexadecyltrimethyl ammonium bromide, methyltrioctyl ammonium chloride, benzyltributyl ammonium chloride and tetrabutyl ammonium bisulfate. A preferred phase transfer catalyst is tetrabutyl ammonium bromide.

The compound of the formula (II), preferably wherein X is a protected keto group, may be N-demethylated or both N-demethylated and O-demethylated by reaction with lithium trialkylborohydride following reaction with ACE-Cl.

Suitable lithium trialkylborohydrides include lithium triethylborohydride, lithium tripropylborohydride, lithium tributylborohydride or lithium tripentylborohydride. A preferred reagent is lithium tri-sec-butylborohydride (sometimes referred to as lithium selectride).

The reaction is carried out in an aprotic solvent such as tetrahydrofuran, diethylether, toluene, dichloromethane, acetonitrile or the like. A non-extreme temperature is generally employed, for example from ambient temperature (about 20-25° C.) or an elevated temperature of 50-70° C., for example at the reflux temperature of the solvent employed.

N-demethylation (via N-decarboxymethylation) may occur first. If the reaction is allowed to proceed, O-demethylation can then occur to yield the N,O-didemethylated product of the formula (III) or a pharmaceutically acceptable salt thereof.

N-alkylation Reaction

By Reductive Alkylation (Route (a))

The compound of the formula (III) (or a pharmaceutically acceptable salt thereof) may be converted to a compound of the formula (I) (or a pharmaceutically acceptable salt thereof) by reaction with an aldehyde (RCHO) (VI) and reduction of the intermediate iminium ion (VII) with a suitable reducing agent.

When the 6-keto function is protected, standard hydride reducing agents may be employed. Suitable reducing agents include lithium aluminium hydrides and alkali metal borohydrides. When the 6-keto function is not protected, milder reducing agents are required, for example a triacetylborohydride such as lithium or sodium triacetoxyborohydride, sodium cyanoborohydride or even hydrogen gas with a catalyst such as palladium. A preferred reagent is sodium triacetoxyborohydride.

The reductive amination reaction is preferably carried out in a solvent such as tetrahydrofuran, dimethylformamide, dimethylsulfoxide, dichloromethane, 1,2-dichloroethane, ethanol, isopropanol or the like. A preferred solvent is 1,2-dichloroethane.

The initial reaction between the aldehyde and secondary amine may take place at ambient temperature, for example 20-25° C., optionally in the presence of molecular sieves and preferably under anhydrous conditions.

Generally, when X is O, the reduction reaction is carried out at a depressed temperature, for example −20° C. to −30° C. When X is a keto protecting group higher temperatures, for example 0-25° C. may be employed.

The compound of formula (I) may be obtained from solution by freeze drying if desired.

By Direct N-alkylation (Route (b))

The conversion of the compound of the formula (III) (or a pharmaceutically acceptable salt thereof) into compound of formula (I) (or a pharmaceutically acceptable salt thereof) by direct alkylation using a compound of the formula Q-CH₂R may be best effected at elevated temperatures, such as 40 to 100° C., 50 to 90° C., 50 to 85° C., 60 to 80° C. and preferably 70 to 80° C.

Suitable alkylating reagents include compounds of the following formula Q-CH₂—R wherein R is as defined in relation to the compound of formula (I) and Q is Cl, Br, OSO₂PhMe or OSO₂Me. Preferable reagents are alkyl halides, such akylhalides include cyclobutylmethyl bromide, cyclobutylmethyl chloride, allylbromide, and allylchloride. Most aptly, the reagent is cyclopropylmethylbromide.

The reaction may be carried out in a solvent such as tetrahydrofuran, ethanol, isopropanol, dimethylformamide, 1-2-dichloroethane and acetonitrile. A preferred solvent is acetonitrile.

The reaction may be carried out in the presence of a phase transfer catalyst, for example a crown ether. Examples of crown ethers include 15-crown-5 and 18-crown-6. A preferred crown ether is 18-Crown-6.

This reaction may be carried out at a low dilution, for example, 0.1-0.3M. A preferred concentration is between 0.2M and 0.3M, for example, 0.25M.

The reaction may also, for example, be carried out in the presence of potassium hydrogen carbonate, sodium hydrogen carbonate, potassium carbonate and sodium carbonate, preferably potassium hydrogen carbonate.

After work up and precipitation, compound (IV) or a pharmaceutically acceptable salt thereof is typically isolated in 85% yield and 96.5% purity.

The following examples illustrate the invention.

### EXAMPLE 1

### N-Demethylation of Oxycodone

Oxycodone free base (1.19 g) was dissolved in 6 ml DCM and Na₂CO₃ (1.60 g) was added. ACE-Cl (1.56 ml) was added drop-wise to the stirred suspension at room temperature (RT) and the reaction mixture was heated to reflux and stirred for 24 hours. The reaction mixture was filtered and the precipitate was washed with DCM. The filtrate was evaporated to dryness. MeOH (20 ml) was added and the mixture stirred for 1 h at RT. The solution was again evaporated to dryness and added water (25 ml) and conc. HCl (1 ml). The aqueous phase was washed twice with DCM and then added ammonia until pH 11. The aqueous phase was extracted five times with DCM:MeOH mix (80:20). The combined phases from the last extraction was dried and evaporated. Crude noroxycodone was obtained as a white foam (0.73 g, 64%), purity 90% by HPLC.

### EXAMPLE 2

### N-Alkylation of noroxycodone

Noroxycodone (0.1 g) and cyclopropanecarboxaldehyde (0.023 g) were mixed in dichloromethane (20 ml) at room temperature for 30 minutes. The solution was cooled to −30° C. and sodium triacetoxyborohydride (0.070 g) was added. The reaction mixture was quenched with sodium bicarbonate solution (20 ml) and the phases were separated. The organic phase was dried (Na₂SO₄), filtered and the solvent removed under reduced pressure to yield crude 3-methyl-naltrexone (0.100 g).

### EXAMPLE 3

### O-Demethylation of N-Cyclopropylmethyl noroxycodone

N-Cyclopropylmethyl noroxycodone (0.20 g, 0.56 mmol) is dissolved in toluene (3 ml) under nitrogen and the reaction flask is immersed in an ice-water bath. Boron tribromide (2.7 eq, 1.5 ml of a 1 M solution in DCM) is added slowly with stirring. The reaction flask is left in the ice-water bath and the temperature is allowed to rise slowly to RT. The reaction mixture is left stirring at RT for 3 hours, after which all of the starting material is consumed. Water (3 ml) is added and the reaction mixture is refluxed for 7 hours. The reaction mixture is basified (NH₄OH, pH 10) and extracted 4 times with DCM. The combined organic extracts are dried (Na₂SO₄), filtered and concentrated under reduced pressure to yield crude naltrexone as a beige solid.

### EXAMPLE 4

### O-Demethylation of N-cyclopropylmethyl noroxycodone hydrochloride

N-cyclopropylmethyl noroxycodone hydrochloride (200 mg, 0.51 mmol) was dissolved in DCM (2 ml) and cooled to 0° C. Boron tribromide (1 M in DCM, 2.55 ml, 2.55 mmol) was added, and the reaction mixture was stirred under inert atmosphere while the temperature was allowed to reach room temperature. HPLC showed that the reaction was fast. Water was added, and the mixture was stirred for 2 h. Additional water and DCM were added, and the pH was adjusted to 10 with aqueous ammonia. The layers were separated, and the aqueous phase was extracted twice with DCM. Drying (MgSO₄) and concentration of the combined organic layers afforded crude Naltrexone (140 mg, 80% yield) as a grey solid.

### EXAMPLE 5

### O-demethylation of oxycodone

Oxycodone HCl (3.04 g, 8.66 mmol) was suspended in DCM (30 ml) under nitrogen and the reaction flask was immersed in an ice-water bath. Boron tribromide (˜3 eq, 25 ml of a 1 M solution in DCM) was added slowly with stirring. The reaction flask was left in the ice-water bath and the temperature rose slowly to room temperature (RT). Stirring at RT was continued overnight after which HPLC indicated full conversion of starting material. Water (25 ml) was added and the bi-phased reaction mixture was refluxed for 1 hour. The reaction mixture was allowed to cool slowly to RT and a white crystalline solid formed. The solid was filtered off and the resulting filtrate was basified (NH₄OH, pH 10). The organic and aqueous phases were separated and the aqueous phase was extracted 5 times using DCM. The combined organic extracts were dried (Na₂SO₄), filtered and the solvent was removed under reduced pressure. The residue was dried under vacuum overnight to yield crude oxymorphone as a beige solid 2.34 g (90% yield), purity <96% by HPLC.

### EXAMPLE 6

### O-Demethylation of noroxycodone

Noroxycodone (0.20 g, 0.66 mmol) was suspended in DCM (3 ml) under nitrogen and the reaction flask was immersed in an ice-water bath. Boron tribromide (˜3 eq, 2 ml of a 1 M solution in DCM) was added slowly with stirring. The reaction flask was left in the ice-water bath and stirring continued while the temperature rose slowly to RT, after which HPLC showed that all of the starting material had been consumed. Water (3 ml) was added and the reaction mixture was refluxed for 7 hours. The reaction mixture was basified (NH₄OH, pH 10) and extracted 4 times with DCM. The product remained in the aqueous phase.

### EXAMPLE 7

### N-Demethylation of Oxycodone

Oxycodone free base (1.19 g) was dissolved in 6 ml DCM and Na₂CO₃ (1.60 g) was added. ACE-Cl (1.56ml) was added drop wise to the stirred suspension at RT, and the reaction mixture was heated to reflux and stirred for 24 hours. The reaction mixture was filtered and the precipitate was washed with DCM. The filtrate was evaporated to dryness. MeOH (20 ml) was added and the mixture stirred for 1 h at RT. The solution was again evaporated to dryness and added water (25 ml) and conc. HCl (1 ml). The aqueous phase was washed twice with DCM and then added ammonia until pH 11. The aqueous phase was extracted five times with DCM:MeOH (80:20). The combined organic extracts were dried (Na₂SO₄), filtered and the solvent was removed under reduced pressure. Crude noroxycodone was obtained as a white foam (0.73 g, 64%), purity 90% by HPLC.

### EXAMPLE 8

### N-cyclopropylmethylation of noroxymorphone

Noroxymorphone (0.100g) and cyclopropylcarboxaldehyde (0.023 g) are mixed in dichloromethane at room temperature. Also after 30 minutes the solution is cooled to −30° C. and NaBH(OAc)₃ added. This reaction is left for two days. HPLC is used to show the presence of naltrexone.

### EXAMPLE 9

### N-cyclopropylmethylation of noroxymorphone

Noroxymorphone (0.1 g) and cyclopropanecarboxaldehyde (0.023 g) are mixed in dichoromethane 20 ml) at room temperature for 30 minutes. The solution is cooled to −30° C. and sodium triacetoxyborohydride (0.07 g) is added. The reaction mixture is quenched with sodium bicarbonate solution (20 ml) and the phases separated. The solution is adjusted to neutrality. The organic phase is dried (Na₂SO₄), is filtered and the solvent is removed under reduced pressure to yield naltrexone.

### EXAMPLE 10

### N-demethylation of oxycodone to Yield noroxycodone HCl

To a mixture of oxycodone (58.5 g), sodium carbonate (37.1 g) and TBAB (5.8 g) in acetonitrile (300 ml) in a 1 l reactor kept at 25° C., ACE-Cl (101 ml) was added. The reaction mixture was stirred at 25° C. for 6 hours after which another portion of sodium carbonate (37.1 g) was added. Stirring was continued for 18 hours. The inorganic base was removed by filtration and the filter cake was washed with isopropanol (2×200 ml) and the filtrate was transferred to a 6 l reactor kept at 20° C. Isopropanol (1400 ml) and water (60 ml) was added and the reaction mixture was left stirring at 25° C. for 22 hours and then 23 hours at 0° C. to ensure complete precipitation of the product. The resulting solid was filtered and dried to yield noroxycodone HCl (35.8 g, 57%) as a white solid, 94% pure by HPLC.

### EXAMPLE 11

### N-demethylation of oxycodone to Yield noroxycodone HCl

To a mixture of oxycodone (60 g), sodium carbonate (2 eq, 40.3 g) and TBAB (6 g) in acetonitrile (300 ml) in a 1 l reactor kept at 25° C., ACE-Cl (103 ml) was added. The reaction mixture was stirred at 25° C. for 20 hours after which another equivalent sodium carbonate (20.2 g) was added. After another 5.5 hours a fourth equivalent of sodium carbonate (20.2 g) was added and stirring was continued for 4 hours. The inorganic base was removed by filtration and the filter cake was washed with isopropanol (500 ml) and the filtrate was transferred to a 6 l reactor kept at 20° C. Isopropanol (1000 ml) and water (40 ml) was added and the reaction mixture was left stirring at 20° C. for 24 hours to ensure complete precipitation of the product. The resulting solid was filtered and dried to yield noroxycodone HCl (41.7 g, 65%) as a white solid >98% pure by HPLC.

### EXAMPLE 12

### N-demethylation of oxycodone to Yield noroxycodone HCl

To a mixture of oxycodone (119 g), sodium carbonate (1 eq, 40.3 g) and TBAB (12 g) in acetonitrile (600 ml) in a 2 l reactor kept at 25° C., ACE-Cl (206 ml) was added. At this point another equivalent of sodium carbonate (40.3 g) was added as well. The reaction mixture was stirred at 25° C. for 18 hours after which another equivalent sodium carbonate (40.3 g) was added. After another 4 hours a fourth equivalent of sodium carbonate (40.3 g) was added and stirring was continued for 3 hours. The inorganic base was removed by filtration and the filter cake was washed with isopropanol (1000 ml) and the filtrate was transferred to a 6 l reactor kept at RT. Isopropanol (2000 ml) and water (96 ml) was added and the reaction mixture was left stirring at RT for 17 hours and at 5° C. for 3 hours to ensure complete precipitation of the product. The resulting solid was filtered and dried to yield noroxycodone HCl (92.3 g, 72%) as a white solid, 98% pure by HPLC.

### EXAMPLE 13

### Direct alkylation of noroxycodone HCl to Yield 3-methyl Naltrexone HCl

To a mixture of noroxycodone HCL (30 g), potassium hydrogen carbonate (43.6 g) and 18-crown-6 (1.4 g) in a 1 l reactor, acetonitrile (348 ml) was added. The temperature was set to 75° C. and cyclopropylmethyl bromide (23.5 g) was added. The resulting mixture was left stirring under nitrogen for 24 hours. The temperature was lowered to 20° C. and the inorganic base was removed by filtration through a silica plug. The silica plug was thereafter washed with acetonitrile (250 ml) and 12 M HCl (10 ml) was added to the solution. Following this, the solution was distilled/concentrated down to a volume of approx. 200 ml. While continuing the distillation of the remaining acetonitrile, toluene (500 ml) was added portion wise to yield a white suspension. The reaction mixture was left stirring at 25° C. for 18 hours after which the resulting solid was filtered and dried to yield 3-methyl Naltrexone HCl (29.5 g, 85%) as a white solid, >96% pure by HPLC.

### EXAMPLE 14

### Direct alkylation of noroxycodone HCl to Yield 3-methyl Naltrexone HCl

To a mixture of noroxycodone HCL (26 g) and potassium hydrogen carbonate (38.5 g) in a 1 l reactor, acetonitrile (320 ml) was added. The temperature was set to 75° C. and cyclopropylmethyl bromide (15.6 g) was added. The resulting mixture was left stirring under nitrogen for 21 hours. The temperature was lowered to 20° C. and the inorganic base was removed by filtration. The filtercake was washed with acetonitrile (200 ml) and conc. HCl (9.4 ml) was added to the solution. Following this, the acetonitrile was removed under reduced pressure. Ethyl acetate (200 ml) was added and the reaction mixture was left stirring at RT for 1 hour. The resulting solid was filtered off and dried to yield 3-methyl Naltrexone HCl (24.5 g, 81%) as a white solid, 90% pure by HPLC.

1. A process for the preparation of a compound of the formula (I) or a pharmaceutically acceptable salt thereof, for example the HCl salt: 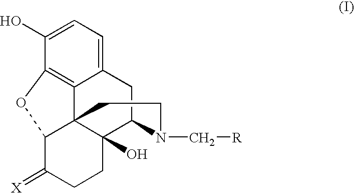
wherein R is a cyclopropyl, cyclobutyl or vinyl group and X is O, CH₂ or diC₁₋₄alkoxy group (optionally linked), which process comprises:
(i) the N-demethylation and optionally the O-demethylation of a compound of the formula (II): 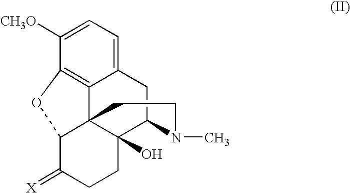
wherein X is as defined in relation for formula (I) to yield a compound of the formula (III) or a pharmaceutically acceptable salt thereof, for example the HCl salt: 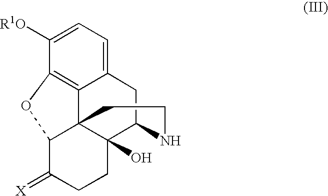
wherein X is as defined in relation to formula (I) and R¹ is CH₃ if only the N-demethylation step is carried out or is H if both the N- and O-demethylation steps are carried out; followed by
(ii) reaction of the compound of formula (III) or a pharmaceutically acceptable salt thereof, with either
(a) a compound of the formula R—CHO where R is as defined in relation to formula (I), followed by reduction of the iminium ion double bond of the resulting intermediate, or
(b) a compound of the formula Q-CH₂R wherein R is as defined in relation to the compound of formula (I) and Q is Cl, Br,OSO₂PhMe or OSO₂Me in the presence of a phase transfer catalyst;
to form a compound of the formula (IV) or a pharmaceutically acceptable salt thereof, for example HCl salt: 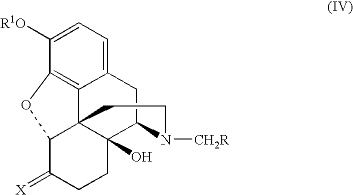
wherein X and R are as defined in relation to the compound of the formula (I) and R¹ is defined as in relation to the compound of formula (III);
(iii) and if R¹ is CH₃, followed by reaction of the compound of formula (IV) or a pharmaceutically acceptable salt thereof, with BBr₃ or other reagents capable of demethylating an aryl methyl ether, and optionally
(iv) obtaining the free base or pharmaceutically acceptable salt thereof as desired.

2. A process as claimed in claim 1 for the preparation of a compound of the formula (I) as claimed in claim 1 or a pharmaceutically acceptable salt thereof: 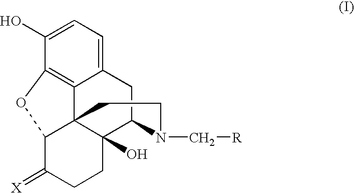
wherein R is a cyclopropyl, cyclobutyl or vinyl group and X is O, CH₂ or diC₁₋₄alkoxy group (optionally linked), which process comprises:
(i) the N-demethylation of a compound of the formula (II): 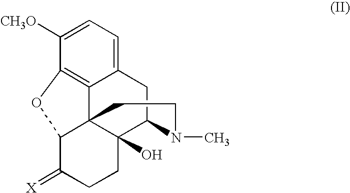
wherein X is as defined in relation for formula (I) by reaction with α-chloroethylchloroformate to yield a compound of the formula (III) or a pharmaceutically acceptable salt thereof, for example the HCl salt: 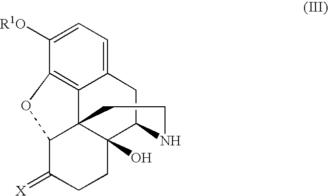
wherein X is as defined in relation to formula (I) and R¹ is CH₃, followed by
(ii) reaction of the compound of formula (III) or a pharmaceutically acceptable salt thereof, with either
(a) a compound of the formula R—CHO where R is as defined in relation to formula (I), followed by reduction of the iminium ion double bond of the resulting intermediate, or
(b) with a compound of the formula Q-CH₂R wherein R is as defined in relation to the compound of formula (I) and Q is Cl, Br,OSO₂PhMe or OSO₂Me in the presence of a phase transfer catalyst;
to form a compound of the formula (IV) or a pharmaceutically acceptable salt thereof, for example a HCl salt: 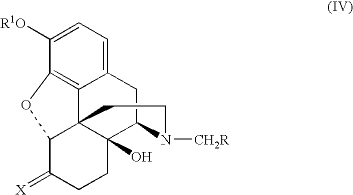
wherein X and R are as defined in relation to the compound of the formula (I) and R¹ is CH₃,
followed by:
(iii) reaction of the compound of formula (IV) or a pharmaceutically acceptable salt thereof, with BBr₃ or other reagents capable of demethylating an aryl methyl ether, and optionally
(iv) obtaining the free base or pharmaceutically acceptable salt thereof as desired.

3. A process as claimed in claim 1 for the preparation of a compound of the formula (I) as claimed in claim 1 or a pharmaceutically acceptable salt thereof: 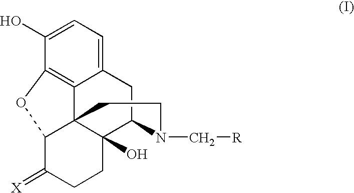
wherein R is a cyclopropyl, cyclobutyl or vinyl group and X is O, CH₂ or diC₁₋₄alkoxy group (optionally linked), which process comprises:
(i) the N-demethylation and O-demethylation of a compound of the formula (II): 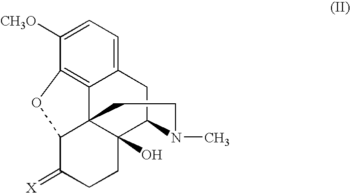
wherein X is as defined in relation for formula (I) to yield a compound of the formula (III) or a pharmaceutically acceptable salt thereof, for example the HCl salt: 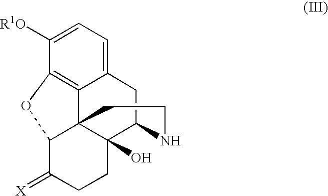
wherein X is as defined in relation to formula (I) and R¹ is H, followed by
(ii) reaction of the compound of formula (III) or a pharmaceutically acceptable salt thereof, with either
(a) a compound of the formula R—CHO where R is as defined in relation to formula (I), to yield on intermediate containing the ion of formula (VII): 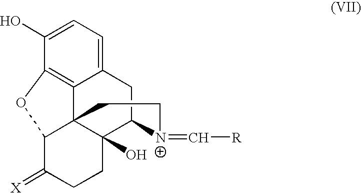
followed by reduction of the iminium ion double bond of the resulting intermediate (VII); or
(b) with a compound of the formula Q-CH₂R wherein R is as defined in relation to the compound of formula (1) and Q is Cl, Br, or OSO₂PhMe or OSO₂Me, in the presence of a phase transfer catalyst and optionally
(iii) obtaining the free base or pharmaceutically acceptable salt thereof as desired.

4. A process as claimed in claim 1 wherein X is OCH₂CH₂O.

5. A process as claimed in claim 1 wherein X is O.

6. A process as claimed in claim 1 wherein the N-demethylation step is performed in an aprotic solvent, preferably dichloromethane or acetonitrile.

7. A process as claimed in claim 6 wherein a carbonate or bicarbonate proton acceptor is present in the N-demethylation step.

8. A process as claimed in claim 1 wherein the N-demethylation step employs an α-chloroethylchoroformate followed by hydrolysis of the resulting intermediate to effect N-demethylation.

9. A process as claimed in claim 1 wherein the N-demethylation step employs a phase transfer catalyst, for example, tetrabutylammonium bromide, hexadecyltrimethyl ammonium bromide, methyltrioctyl ammonium chloride, benzyltributyl ammonium chloride and tetrabutyl ammonium bisulfate, preferably tetrabutyl ammonium bromide.

10. A process as claimed in claim 1 wherein step (iia) is performed at a depressed temperature for example 5° C. to −30° C.

11. A process as claimed in claim 1 wherein step (iib) is performed at a raised temperature for example 50° C. to 85° C.

12. A process as claimed in claim 1 wherein the reducing agent used in step (iia) may be a triacetoxyborohydride for example sodium triacetoxyborohydride, a cyanoborohydride, for example sodium cyanoborohydride or hydrogen and a catalyst, for example palladium.

13. A process as claimed in claim 1 wherein step (iia) is performed a solvent such as tetrahydrofuran, ethanol, isopropanol, dimethylformamide or 1,2-dichloroethane

14. A process as claimed in claim 13 wherein step (iia) is performed in 1,2-dichloroethane.

15. A process as claimed in claim 1 wherein step (iib) is performed in acetonitrile.

16. A process as claimed in claim 1 wherein in step (iib) Q is a halide group, preferably Br.

17. A process as claimed in claim 1 wherein in step (iib) the phase transfer catalyst is a crown ether.

18. A process as claimed in claim 1 wherein R is cyclopropyl.

19. A process as claimed in claim 1 wherein the O-demethylation steps in either step (i) or step (iii) is effected using BBr₃.

20. A process as claimed in claim 1 wherein step (iii) is performed in an aprotic solvent, such as toluene, tetrahydrofuran, chloroform, dichloromethane or 1,2-dichloroethane.

21. A process as claimed in claim 20 wherein BBr₃ is added at a depressed temperature.

22. A process as claimed in claim 1 for the preparation of a compound of the formula (III) as defined in claim 1 by the N-demethylation of a compound of the formula (II) as defined in claim 1 by reaction with α-chloroethylchloroformate.

23. A process as claimed in claim 17 wherein the crown ether is 18-crown-6.

24. A process as claimed in claim 1 where the N-demethylation and O-demethylation (step i) is effected using an aryl or alkyl chloroformate, preferably using α-chloroethylchloroformate, followed by hydrolysis of the resulting intermediate, preferably using lithium tri-sec-butylborohydride.

25. A process as claimed in claim 1 where the N-demethylation is effected using an aryl or alkyl chloroformate followed by hydrolysis of the resulting intermediate.

26. A process as claimed in claim 25 which employs α-chloroalkyl chloroformate, preferably α-chloroC₁₋₆chloroformate.

27. A process as claimed in claim 26 wherein the α-chloroC₁₋₆chloroformate is α-chloroethylchloroformate.

28. A process as claimed in claim 27 wherein the hydrolysis is effected using an alcohol such as methanol, aqueous tetrahydrofuran, lithium tri-sec-butylborohydride, or aqueous isopropanol.

29. A process as claimed in claim 25 which employs a C₂₋₆chloroformate, for example ethylchloroformate.

30. A process as claimed in claim 29 wherein the hydrolysis is effected using tetrahydrofuran or aqueous isopropanol.

31. A process as claimed in claim 1 wherein the reaction solvent in the N-demethylation and O-demethylation reaction is selected from tetrahydrofuran, acetonitrile, dimethylformamide, dichloromethane or 1,2-dichloroethane, preferably dichloromethane or acetonitrile.
